(19) United States
(12) Patent Application Publication
(10) Pub. No.: US 2017/0204474 A1
(43) Pub. Date: Jul. 20, 2017

(54) Bulk Allele Discrimination Assay

(71) Applicant: General Mills, Inc., Minneapolis, MN (US)

(72) Inventors: Eric Wayne Jackson, Wamego, KS (US); Ryan Hal Brown, Concord, NC (US)

(21) Appl. No.: 15/326,358

(22) PCT Filed: Jul. 16, 2015
(86) PCT No.: PCT/US15/40763

§ 371 Date: Jan. 13, 2017

Related U.S. Application Data
(60) Provisional application No. 62/025,327, filed on Jul. 16, 2014.

(51) Int. Cl. C12Q 1/68

(52) U.S. Cl. CPC C12Q 1/6895; C12Q 2600/156; C12Q 2600/16; C12Q 2600/13

(57) ABSTRACT

A method is described for bulk allele discrimination of multiple single nucleotide polymorphisms in multiple individuals. Also described is a kit for use in performing a method for bulk allele discrimination of multiple single nucleotide polymorphisms in multiple individuals.

### CROSS-REFERENCE TO RELATED APPLICATIONS

The instant application claims priority to U.S. Provisional Application No. 62/025,327, filed Jul. 16, 2014, including appendices to the specification titled “Table1forfiling7934US01.pdf” and “Table2forapplication7934US01forfiling.pdf” which are incorporated by reference in their entireties herein.

### SEQUENCE LISTING

The instant application contains a Sequence Listing which has been submitted electronically in ASCII format and is hereby incorporated by reference in its entirety. Said ASCII copy, created on Jul. 16, 2015, is named 7934WO01_SequenceListing.txt and is 10,130,979 bytes in size. In addition, the application includes sequence listing tables titled “Table1forfiling7934WO01.pdf” and “Table2forfiling7934WO01.pdf” which are incorporated by reference in their entireties herein.

### BACKGROUND

Advances in nucleic acid sequencing technologies have led to determining the sequence and structure of individual genes and entire genomes in a wide variety of organisms. Although a single genome is shared amongst individuals in a species, genetic variation, such as single nucleotide polymorphisms, provide for genetic differences between individual organisms. Single nucleotide polymorphisms, or SNPs, or other polymorphisms can sometimes be linked to traits that vary amongst the individuals in a population of the same species. While recent techniques have taken advantage of the link between genetic variation and trait variability in order to make advances in understanding plant and animal traits and diseases, there remains a need for techniques that provide fast, accurate, high throughput, and cost effective ways to identify allele specific variations within individuals of a population.

### SUMMARY

The present disclosure relates to a method for bulk allele discrimination in individuals within a population.

In one embodiment, a method includes the steps of providing a plurality of primer pairs, each primer pair of the plurality of primer pairs being configured to amplify a unique locus in a pseudo genome, the pseudo genome comprising at least a portion of a full genome and including a plurality of identified polymorphisms; for each of a plurality of individuals, subjecting a sample comprising genomic DNA (gDNA) to a polymerase chain reaction using at least a portion of the plurality of primer pairs to obtain a pool of amplified products for each of the plurality of individuals; determining sequences for each of the amplified products; and determining an allele composition of each individual of the plurality of individuals based on the determined sequences, wherein each unique locus has at least two possible alleles.

In some embodiments, the at least a portion of the plurality of primer pairs includes at least 100 primer pairs.

In some embodiments, the method can further include attaching a tag to at least one end of the amplified products in each pool to produce a plurality of coded pools, the tag being different for each pool of amplified products, and combining the coded pools prior to determining the sequences of the amplified products.

In some embodiments, the identified polymorphisms can be single nucleotide polymorphisms (SNPs).

In some embodiments, the determined sequences can be 1 to 12 polymorphic nucleotides per unique locus.

In some embodiments, the average number of polymorphic nucleotides per unique locus of the determined sequences can be greater than the average number of identified polymorphisms per unique locus.

In some embodiments, the method can further include determining the zygosity of each individual of the plurality of individuals at each unique locus.

In some embodiments, the method can further include determining linkage disequilibrium of the identified polymorphisms using the at least a portion of the plurality of primer pairs; selecting a subset of the primer pairs based on the linkage disequilibrium; and using the subset of primer pairs to obtain the pool of amplified products.

In some embodiments, the method can further include providing a plurality of additional primer pairs, the plurality of additional primer pairs configured to amplify a plurality of additional loci on one or more selected genes in the full genome and outside of the pseudo genome. In some embodiments, the plurality of additional loci can be on an avenanthramide gene. In some embodiments, the plurality of additional loci can be on two or more genes associated with a biosynthetic pathway. In some embodiments, the biosynthetic pathway can be a beta-glucan synthesis pathway.

In some embodiments, the full genome can be representative of a species. In some embodiments, the full genome can be representative of a selected population within a species. In some embodiments, the selected population can be a breed or strain within a species.

In some embodiments, the full genome can be an oat, a broccoli, or a maize genome.

In some embodiments, the pseudo genome contains at least a portion of each chromosome in the full genome. In some embodiments, the pseudo genome can be a transcriptome.

Also provided herein is a method for producing a library of primers for genotyping a plurality of individuals. The method includes providing a pseudo genome sequence, the pseudo genome sequence being derived from a full genome sequence and including a plurality of identified polymorphisms; designing primer pairs configured to amplify a locus containing each identified polymorphism, each primer pair configured to amplify a unique locus in the pseudo genome sequence; synthesizing at least a portion of the primer pairs; determining linkage disequilibrium of the identified polymorphisms using the primer pairs; selecting a subset of the primer pairs based on the linkage disequilibrium; and using the subset of primer pairs to produce the library.

In some embodiments, the library includes at least 100 primer pairs.

In some embodiments, the identified polymorphisms can be SNPs.

In some embodiments, the method can further include providing a plurality of additional primer pairs, the plurality of additional primer pairs configured to amplify a plurality of additional loci on one or more selected genes in the full genome and outside of the pseudo genome.

In some embodiments, the plurality of additional loci can be on two or more genes associated with a biosynthetic pathway. In some embodiments, the plurality of additional loci can be on an avenanthramide gene. In some embodiments, the biosynthetic pathway can be a beta-glucan synthesis pathway.

In some embodiments, the full genome can be representative of a species. In some embodiments, the full genome can be representative of a selected population within a species. In some embodiments, the selected population can be a breed or strain within a species.

In some embodiments, the pseudo genome sequence contains at least a portion of each chromosome in the full genome sequence. In some embodiments, the pseudo genome sequence can be a transcriptome sequence.

In some embodiments, the full genome sequence can be an oat, a broccoli, or a maize genome sequence.

These and various other features and advantages will be apparent from a reading of the following detailed description.

### BRIEF DESCRIPTION OF DRAWINGS

FIG. 1 is a graph showing the distribution of loci based on the percentage of missing data for 15 tested oat lines using bulk allele discrimination (dark bars) and genotype by sequencing (light bars).

FIG. 2 is a set of graphs showing the genome distribution of loci for which polymorphic nucleotides were detected using bulk allele discrimination (top graph) as compared to the oat genome assembly (bottom graph). Dashed lines in the top graph represent delineation of linkage blocks.

### DETAILED DESCRIPTION

The present disclosure relates to methods and compositions related to bulk allele discrimination in individuals within a population. In particular, the present disclosure relates to the determination of allele composition in a plurality of individuals over a plurality of loci. Individuals can be of any species, including plant (e.g., oat, maize, broccoli, and the like), animal (e.g., cattle, horses, swine, and the like), insect, and microbial species.

Many previously known methods of determining allele composition of individuals rely on the use of probe or primer arrays on chips, which can allow for the determination of allele composition of a single individual per chip over a plurality of loci. Such previously known methods thus require a chip for each individual to be prepared, increasing the amount of time required for preparation as the number of individuals to be tested increases, as well as time required for reading of the chips for all individuals to be tested. In contrast, the present methods use multiplexed polymerase chain reaction (PCR) and high throughput sequencing to determine allele composition in a plurality of individuals over a plurality of loci in a single assay, which can be completed within a few hours to a few days.

Previously known methods of determining allele composition of individuals that rely on chip technology generally have a relatively high cost when determining allele composition of a plurality of individuals because a chip is required for each individual to be tested. In contrast, methods disclosed herein can reduce cost by determining allele composition in a plurality of individuals using standard PCR equipment and multi well plates to produce a pool of amplified products for each individual in a multi well PCR plate. In some embodiments, cost for performing the presently provided methods can also be reduced by combining pools of amplified products and sequencing in a single high throughput sequencing reaction.

For previously known methods of determining allele composition of individuals that rely on hybridization of a probe to a locus, the presence of multiple polymorphic nucleotides, rather than a single polymorphic nucleotide, can result in failure of the probe to properly hybridize to the locus and no determination of allele composition at that locus. In some previously known methods, allele composition is determined by enzymatic detection of a known single nucleotide polymorphism (SNP). The presence of multiple polymorphic nucleotides can often not be detected or can result in a failure of detection of any polymorphic nucleotides. In some previously known methods, a primer that incorporates a single polymorphic nucleotide is used. In the present methods, however, a library of primers is designed to flank known polymorphic nucleotides, but not include the polymorphic nucleotide, allowing for multiple polymorphisms at a single locus to be detected in a single assay.

Primers for use in the present methods can be designed a plurality of unique loci, with each unique locus having a known polymorphic nucleotide located near the center of the unique locus. Thus, the present methods are less likely to be disrupted by unexpected polymorphic nucleotides than previously known methods that incorporate a known polymorphic nucleotide in a probe or primer, or rely on an enzyme that detects a SNP. Furthermore, the present methods include a high throughput sequencing reaction which can provide sequence information for more than one polymorphic nucleotide in a unique locus.

In some embodiments, the presently disclosed methods and compositions can provide greater reproducibility for determining allele composition over previously known methods. For example, in some previously known methods for determining allele composition, genomic DNA is cut with a restriction enzyme, where a polymorphism results in creation or destruction of restriction enzyme sites. Allele composition is then determined by examining the fragments resulting from restriction enzyme cutting (e.g., by measuring length, mass, or by sequencing). Such previously known methods are often not consistent between different populations within a species because restrictions sites may be more or less common in each population. Further, such previously known methods cannot be used to target a specific locus within the genome since restriction sites are randomly distributed throughout the genome. The present methods, by contrast, can specifically target multiple loci within a genome to consistently determine allele composition over the targeted loci.

Allele composition of an individual organism can provide information about the phenotype of the individual. A phenotype can include any observable trait, including without limitation, morphology, development, biochemical or physiological properties, behavior, and the like. By using a population of individuals (i.e., a training set) with known phenotypes and determining the allele compositions of the individuals, in some embodiments, an allele can be statistically associated with a phenotypic trait. Presence in an individual of an allele associated with a phenotypic trait can then be used to predict a likely phenotype of the individual, including morphology, disease resistance or susceptibility, development, and the like. Similarly, because alleles are heritable, allele composition a parent can be used to predict the probability of offspring inheriting traits associated with the allele composition of the parent. Thus, the presently disclosed methods and compositions provide for rapid genome wide specific allele composition analysis, which can be used to take advantage of statistical modelling methods in order to predict phenotypes of individuals within a population, as well as model efficient breeding schemes that are likely to result in offspring with desired phenotypic traits.

Individuals in most species of living organisms can be distinguished from one another by examining the sequence of the DNA of an individual and comparing it to the DNA sequence of another individual. Similarly, highly related individuals in breeds or lines of a population tend to share similar allele compositions, but also tend to have allele compositions that differ more greatly from individuals in other lines or breeds. Thus, the presently disclosed methods and compositions can be used to differentiate individuals within a population or identify an individual as belonging to a line or breed of a population.

Allele composition of an individual organism can also provide information about the overall genotype of the individual. For example, due to the nature of chromosomal crossover, different loci can be linked. Such linkage blocks are generally inherited together. For this reason, a particular allele at one locus that is observed in an individual organism can be indicative of the likely sequence of a different nearby locus in the same individual. In some embodiments, the nucleic acid sequences in a pseudo genome can have known linkage blocks. In other embodiments, a method used herein can be used to identify linkage blocks amongst the nucleic acid sequences in a pseudo genome. A linkage block can be statistically determined based on observed allele compositions in which certain alleles occur together in individuals at a frequency that indicates non-random inheritance.

A “pseudo genome,” as used herein, is a collection of nucleic acid sequences from a plurality of loci from a genome, where each locus includes at least one identified polymorphic nucleotide. A pseudo genome can preferably include at least 2,000 loci. In some embodiments, however, a pseudo genome can include from 20 to 20,000 loci (e.g., from 100 to 15,000, from 500 to 10,000, 2,000 to 8,000, and the like) from a genome. In some embodiments, the nucleic acid sequences in a pseudo genome can have known chromosome locations in the genome (i.e., the nucleic acid sequences can be anchored in a consensus genomic map). In some embodiments, a pseudo genome can include at least a portion of each chromosome of the genome from which it's derived. In some embodiments, each locus in a pseudo genome can be in genes expressed in a specific cell type within the organism. In other words, in some embodiments, a pseudo genome sequence can be a transcriptome sequence.

As used herein, the term “allele” refers to a heritable variant of a nucleic acid sequence at a particular locus in a genome or pseudo genome. Alleles can be distinguished by the identity of one or more polymorphic nucleotides within a locus. A nucleotide can be considered to be polymorphic if the nucleotide differs between members of a species or paired chromosomes. A polymorphic nucleotide can differ in base composition (i.e., adenosine, thymine, guanine, cytosine, uracil, and the like), or can be an insertion or deletion. A locus can contain a single polymorphic nucleotide (SNP) or more than one (e.g., 2, 3, 4, 5, or more) polymorphic nucleotide. Multiple polymorphic nucleotides can be spaced throughout a locus or be grouped to form insertions, deletions, inversions, duplications, and the like, or combinations thereof, within the locus.

A “locus,” as described herein, is a location in a genome. A locus can be found anywhere in a genome (e.g., in a gene or intergenic region). If located in a gene, a locus can be in an intron and/or an exon. In some embodiments, a locus can span different regions of genomic DNA (e.g., genic/intergenic transition and/or intron/exon transition).

A method for determining allele composition can include performing a polymerase chain reaction (PCR) using a sample from each of a plurality of individuals that includes DNA (e.g., gDNA or cDNA) to obtain a pool of amplified products for each of the plurality of individuals. A PCR suitable for use in a provided method can be multiplexed such that a single reaction is configured to amplify multiple loci within the DNA in a sample from an individual. Each multiplexed PCR can contain a plurality of primer pairs (e.g., from 20 to 20,000 primer pairs, from 4000 to 15,000 primer pairs, and the like), with each of the primer pairs configured to amplify a unique locus in a pseudo genome.

As used herein, the term “unique locus” refers to a sequence in a pseudo genome that is amplified by one primer pair, but not any other primer pair in a single PCR. In some embodiments, a unique locus can overlap with another unique locus. A unique locus can contain at least one identified polymorphic nucleotide. A unique locus can have a length of from about 80 nucleotides to about 500 nucleotides in length (e.g., from 80 nucleotides to 200 nucleotides, from 80 nucleotides to 150 nucleotides, from 80 nucleotides to 120 nucleotides, and the like).

By using primer pairs that amplify unique loci in a pseudo genome, a method for determining allele composition provided herein can be performed without the need for a genome that has been fully sequenced, while being able to determine allele composition in a targeted manner. As used herein, a “fully sequenced genome” refers to a genome over which at least 95% of the nucleic acid sequence has been determined. However, sequencing of a genome should be sufficient enough to be able to identify a plurality of loci with polymorphic nucleotides within the genome. In some embodiments, the population of individuals used for sequencing a genome can be representative of one or more populations (e.g., lines or breeds) within a species or can be representative of substantially the entire species.

Primer pairs for use in a method for determination of allele composition in a plurality of individuals over a plurality of loci can be designed to be compatible with one another in a single PCR. As used herein, the term “compatible primer pairs” refers to primer pairs that are each capable of priming nucleic acid amplification of a unique locus under the same PCR conditions. Attributes that can be considered when designing primer pairs to be compatible include, without limitation, melting temperature, GC content, length of amplified fragment, length of the unique locus, sequence similarity between primers in a pair, sequence similarity within a single primer, sequence similarity with other primer pairs and/or loci, and the like. Primer pairs can be designed to be from about 18 nucleotides to about 32 nucleotides in length (e.g., 19 to 30 nucleotides, 20 to 29 nucleotides, 21 to 32 nucleotides, and the like) in order to adjust the compatibility of the primer pairs. Primers pairs can also be designed to amplify unique loci having a length of 80 nucleotides to about 500 nucleotides in length (e.g., from 80 nucleotides to 120 nucleotides, from 80 nucleotides to 150 nucleotides, from 80 nucleotides to 200 nucleotides, and the like) to adjust the compatibility of the primer pairs. In some embodiments, primer pairs for use in a method provided herein can be designed using software that can predict compatibility of primer pairs. Examples of software that can be used to design primer pairs include, for example, Ion AmpliSeq™ Designer (Life Technologies™, Carlsbad, Calif., USA), PrimerPlex (Premier Biosoft, Palo Alto, Calif., USA), MPprimer (biocompute.bmi.ac.cn/MPprimer/), and muPlex (Rachlin et al. (2005) Nucleic Acids Research. 33(Web Server Issue):W544-W547).

In some embodiments, one or more primer pairs designed to amplify additional loci on one or more selected genes in a genome and outside of the pseudo genome can be used in a method provided herein. Primer pairs for additional loci need not be designed to amplify a region having an identified polymorphism. Primer pairs for additional loci can be designed to amplify all or part of a selected gene. For example, a plurality of primer pairs can be used to amplify the entirety of a selected gene, or parts of the selected gene.

Genes outside of a pseudo genome can be selected in order to provide information about a desired trait. For example, a plurality of additional primers designed to amplify loci on a gene associated with a biosynthetic pathway (e.g., beta-glucan synthesis pathway genes, an avenanthramide gene, a tocopherol gene, and the like). In some embodiments, a plurality of additional primers are designed to amplify loci on two or more related genes (e.g., beta-glucan synthesis genes, protein synthesis genes, and the like).

A method for determining allele composition provided herein can use a multiplexing PCR protocol that can amplify the desired number of unique loci (e.g., from 20 to 10,000 loci) in a pseudo genome and any additional loci.

In some embodiments, a PCR protocol suitable for use in a method provided herein can be designed to include features that reduce the presence of PCR artifacts, such as primer dimers, super amplicons, and the like, in a pool of amplified products. For example, primers for primer pairs can be designed with one or more cleavable feature (e.g., a uracil that can be degraded by a uracil DNA glycosylase) to degrade primers and PCR artifacts after amplification. In some embodiments, degradation of primers and PCR artifacts can be followed by adding universal adaptors for further amplification and dilution of PCR artifacts. An example of a kit for performing a PCR protocol that includes features for reducing PCR artifacts includes the Ion AmpliSeg™ Library Kit 2.0 (Life Technologies™).

Following PCR, pools of amplified products from each individual can be tagged in order to produce a plurality of coded pools. A tag suitable for use in a method provided herein can be any moiety that can be used to distinguish one pool of amplified products from another. A tag can be used to distinguish each pool of amplified products before, during, or after sequencing. Examples of appropriate tags include, for example, artificial nucleotide sequences (e.g., molecular barcodes), chromophores, fluorophores, and the like. A tag can be attached to one or both ends of the amplified products in a pool using known techniques, such as DNA ligation.

In some embodiments, one or more adaptor for sequencing can be attached to one or both ends of each amplified product. In some embodiments, an adaptor for sequencing can be an artificial priming site. As used herein, the term “artificial priming site” refers to a nucleotide sequence that primes a sequencing reaction. An artificial priming site can be selected based on a high throughput sequencing protocol to be used.

In some embodiments, an adaptor for sequencing can be an anchor molecule. As used herein, an “anchor molecule” refers to a molecule that binds an amplified product to a substrate. An anchor molecule can be, for example, an artificial nucleic acid sequence or biotin molecule. An anchor molecule can be selected based on a high throughput sequencing protocol to be used.

Pools of amplified products are sequenced using a high throughput sequencing protocol. A high throughput sequencing protocol is a protocol capable of sequencing the pools of amplified products in a single run. In some embodiments, a high throughput sequencing protocol is capable of sequencing combined pools of amplified products in a single run. If the pools are tagged prior to sequencing, the tags can be used to identify which pool (e.g., which individual) each amplified product originated from. Examples of high throughput sequencing protocols include, without limitation, ion semiconductor sequencing (e.g., Ion Torrent™, Life Technologies™) and sequencing by synthesis (e.g., Illumina dye sequencing and pyrosequencing,). In some embodiments, a high throughput sequencing protocol can be chosen based on the multiplex PCR protocol used, the size of the unique loci to be sequenced, and/or the tag used to produce coded pools.

Following sequencing, sequences of amplified unique loci for each of a plurality of individuals can be analyzed. The identity of one or more polymorphic nucleotides can be used to determine which allele or alleles each individual carries in its genome. The allele or alleles in an individual can be determined for an amplified unique locus by comparing a sequence determined by high throughput sequencing for the individual to the pseudo genome used to design the primers for amplification of the unique locus. Zygosity at a particular locus can also be determined by determining how many alleles of the locus an individual carries in its genome.

Once allele composition of a plurality of individuals across a plurality of unique loci is determined, allele composition can be statistically associated with phenotypic traits of the individuals if phenotypic traits of each individual are known, as described above. Once a phenotypic trait is statistically associated with one or more alleles, allele composition of an individual can be used to predict the phenotype of the individual, or predict the likely phenotype of progeny of the individual. In some embodiments, a method provided herein can be used to statistically predict the phenotypes of progeny from individuals in order to design a breeding scheme that results in progeny with desired phenotypic traits.

In some embodiments, sequences of amplified unique loci can be analyzed to determine the number of polymorphic nucleotides in the amplified unique loci. In some embodiments, the number of polymorphic nucleotides that are determined in the amplified unique loci can be greater than the number of polymorphisms identified for use in the pseudo genome used for the method. For example, in some embodiments, a single polymorphic nucleotide can be identified in a locus in a genome, which is used in a pseudo genome. Following sequencing, the amplified unique locus corresponding to the locus in the pseudo genome can be found to have two or more polymorphic nucleotides. Thus, in some embodiments, the average number of polymorphic nucleotides per unique locus of the sequenced loci can be greater than the average number of polymorphic nucleotides per locus in the pseudo genome. An advantage to the ability to identify additional polymorphisms per locus using a method provided herein is that the presence of multiple polymorphisms increases the statistical confidence of the identity of a particular allele. That is, it is less likely that multiple polymorphic nucleotides found in a sequenced locus are a result of an amplification error or a sequencing error than a single polymorphic nucleotide.

In some embodiments, the allele composition of a plurality of individuals over a plurality of loci can be used to statistically determine linkage disequilibrium of alleles. As used herein “linkage disequilibrium” is the non-random inheritance of alleles at two or more loci. Alleles at two or more loci that are inherited disproportionately together are referred to as a linkage group herein.

In some embodiments, determined linkage disequilibrium can be used to design a library of primers having fewer primer pairs than used to determine the linkage disequilibrium. For example, if alleles at two or more loci are known to be inherited together, fewer primer pairs need be used to determine the likely allele composition of all of the loci. In some embodiments, a primer pair with desired traits, such as reliable amplification, the ability to amplify in desired amplification conditions, or is known to amplify a locus with multiple polymorphic nucleotides, can be selected to amplify a locus that represents a linkage group. In some embodiments, a library of primers having fewer primer pairs than the library of primer pairs used to determine linkage disequilibrium can be designed to determine allele composition of individuals with an accuracy similar to that of the library of primer pairs used to determine linkage disequilibrium. Such a library having fewer primer pairs can have an advantage of being less expensive to produce than the larger library.

A kit is provided herein that includes a plurality of primer pairs suitable for performing a method described herein. A kit can further include additional components for use in a method provided herein including, without limitation, PCR reagents (e.g., nucleotides, buffers, and the like), containers for performing one or more step of a method provided herein (e.g., plates, tubes, and the like), reagents for normalizing PCR products, instructions for performing one or more step of a method provided herein, reagents for sequencing, and the like.

The examples provided below are intended to describe particular embodiments of the invention, and are not intended to limit the scope of the invention.

### EXAMPLES

### Example 1

### Oat Bulk Allele Discrimination (BAD) Assay

Three different oat populations representing a general breeding population, a bi-parental mapping population, and a global diversity population were used in this study. The breeding population included 15 F₄-derived lines from the General Mills, Inc. oat breeding program. The mapping population included 94 recombinant inbred lines (RIL). The global diversity set included 96 lines selected from the National Small Grains Collection representing historic cultivars and landraces.

Eight seed from each line per population were planted in a greenhouse. Once the secondary leaves emerged, primary leaves from six plants representing each line were bulk-harvested to produce a composite sample. Genomic DNA was extracted from the composite tissue samples in a 96-well format using a QIAGEN ® (Venlo, Netherlands) DNeasy ® Plant Mini Kit.

SNP targets were identified from expressed sequences (ES) and/or complexity reduced sequences (CRS) as described by Oliver et al. (Oliver et al. 2011. BMC Genomics. 12:77; Oliver et al. 2013. PLOSOne. 8:3). In brief, ES and/or CRS from multiple plants were aligned to identify high quality SNPs. A pseudo genome was constructed by merging 7,506 SNP containing sequences from each line into a composite assembly using ambiguous based codes for variant alleles. SNP containing sequences used for assembly of the pseudo genome are shown in Table 1 (contig sequence) and the attached Sequence Listing as SEQ ID NOS:1-5402. The identified SNP in each contig sequence is shown in italics in Table 1. The pseudo genome was then physically anchored to chromosomes and gene/marker annotations were added using an oat linkage map. In addition, linkage disequilibrium analyses of the breeding and diversity populations were used to annotate the linkage blocks across both populations. The pseudo genome was then used to design 5,406 primers for multiplex PCR using the AmpliSeq™ designer pipeline (www.ampliseq.com/displaychangeLog.action; Life Technologies™, USA). The primers were reviewed to ensure adequate genome and linkage block coverage in order to include primers at least five loci in each of the 136 linkage blocks based on the breeding population. The designed primers were synthesized for use with the Ion AmpliSeq™ Library Kit 2.0 (Life Technologies™). Primers are shown in the attached Sequence Listing as SEQ ID NOS:10805-21616. The sequences in the contig sequences targeted by the primers are in bold in Table 1. The sequence identifiers for the primers for each contig sequence are identified under the columns labeled “BAD Fwd” and “BAD Rev.”

A multiplexed PCR amplification was set up (4 μL of 5× Ion AmpliSeq™ HiFi Master Mix, 4 μL of 5× Ion AmpliSeq™ Primer Pool, 10 ng of DNA and the reaction was brought to 20 μL with water) for each of 215 individual plants and cycled using the following parameters: 99° C. for 2 minutes, 14 cycles of 99° C. for 15 seconds and 60° C. for 8 minutes, ending with a 10° C. hold for up to one hour.

Following amplification, 2 μL of FuPa Reagent was added to each sample to partially digest the primer sequences. The PCR plate was placed in a thermal cycler with the following parameters: 50° C. for 10 minutes, 55° C. for 10 minutes, 60° C. for 20 minutes, ending with a 10° C. hold for up to one hour.

Adapters were ligated to the amplified products by adding 4 μL of switch solution, 2 μL of a diluted barcode adapter mix (4 μL the Ion P1 Adapter, 2 μL of the Ion Xpress™ barcodes of choice, and 4 μL of water) and 2 μL of DNA Ligase. The plate was then placed in a thermal cycler at 22° C. for 30 minutes, 72° C. for 10 minutes, and ending with a 10° C. hold. The amplified products were then purified.

To equalize the samples, either an Equalizer kit (Life Technologies™) or quantitative PCR was used. When the Equalizer kit was used, 50 μL of Platinum®PCR SuperMix High Fidelity and 2 μL of Equalizer™ Primers (Life Technologies™) were added to each bead pellet. The PCR plate was placed in a thermal cycler for a single cycle at 98° C. for 2 minutes, 7 cycles of 98° C. for 15 seconds and 60° C. for 1 minute, ending with a 10° C. hold for up to one hour. After thermal cycling, 10 μL of Equalizer™ Capture was added to each reaction and incubated at room temperature for five minutes. 6 μL of washed beads were added to each plate well, mixed and incubated at room temperature for 5 minutes and then placed in a magnetic rack until all the beads were stuck to the side of the wells and the supernatant was removed. Two washes were performed using 150 μL of Equalizer™ Wash Buffer. After the second wash, 100 μL of Equalizer™ Elution Buffer was added to each reaction and the plate was placed in a thermal cycler at 30° C. for 5 minutes. The supernatants were removed from the beads and placed in a new plate.

Libraries were also normalized using quantitative PCR and sequenced on the Ion Proton™ (Life Technologies™) using a sequencing protocol supplied by the manufacturer.

A preliminary test was done to benchmark the BAD assay against genotype-by-sequencing (GBS). Fifteen lines from the General Mills CropBioscience oat breeding program were selected and DNA was extracted from six F₄-derived plants for each line to capture allele segregates. The lines were then genotyped using a GBS protocol developed by Yung-Fen Huang et al. (in press) and the BAD assay. The probes used for the GBS protocol are provided in the attached Sequence Listing as SEQ ID NOS:5403-10804. The sequences in the contig sequences targeted by the probes are underlined in Table 1. The sequence identifiers for the probe for each contig sequence are identified under the column labeled “GBS probe.” The same 15 lines were then tested using the BAD assay.

Evaluation of the average read counts per locus across all 15 lines tested using the BAD assay ranged from 0.1 to 7,586.8. Over half the loci had average read counts between 10 and 350 per locus. Interestingly, read counts from 19 loci were greater than 1000 reads per locus per line. This suggests that optimization of the normalization process could further improve the number of loci successfully targeted using the BAD assay.

The BAD assay was able to reliably detect one or more polymorphic nucleotides in 1,198 of the contig sequences in the tested population. An additional 3,603 loci could be amplified in at least 30% of the tested lines using the BAD assay, but did not show polymorphism in the tested lines. Of the total number of loci that could be amplified, 2,132 could provide at least 10 reads in all 15 lines. Table 2 shows contig sequences in which the BAD assay detected polymorphic nucleotides. The column in Table 2 labeled “GBS” identifies whether the GBS probe also worked (“Y”) or not (“N”). None of the contig sequences had polymorphic nucleotides that could be successfully detected by GBS probe but not by BAD assay using the tested population.

Table 2 also shows how many polymorphic nucleotides were determined using the BAD assay. Of the contig sequences in which one or more polymorphisms were detected using the BAD assay, 593 had a single nucleotide polymorphism detected, 239 had 2 polymorphic nucleotides detected, 143 had 3 polymorphic nucleotides detected, 97 had 4 polymorphic nucleotides detected, 65 had polymorphic nucleotides detected, 35 had 6 polymorphic nucleotides detected, 20 had 7 polymorphic nucleotides detected, 5 had 8 polymorphic nucleotides detected, and 1 had 9 polymorphic nucleotides detected.

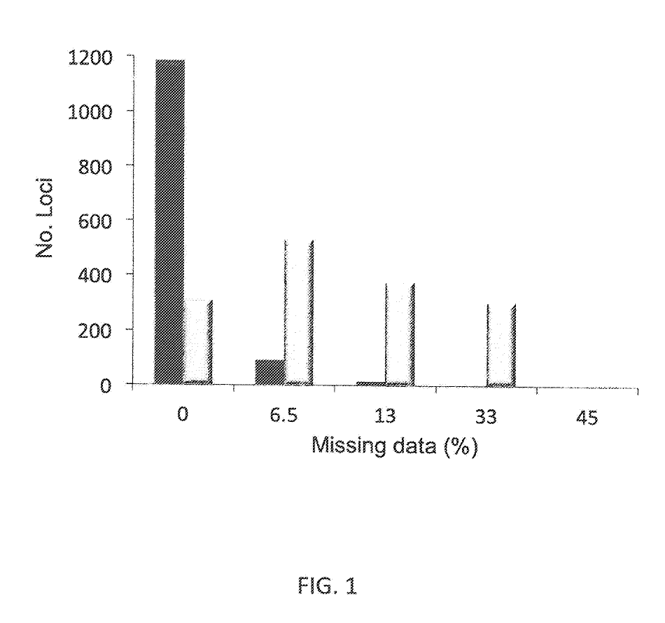

The BAD assay also provided more reliable results. For example, while the BAD assay provided data for all 15 tested lines for about 1200 loci, GBS provided complete data for only about 300 loci. Overall, the BAD assay was able to provide results for about 1300 loci with 6.5% missing data or less, while GBS provided results for approximately 850 loci with 6.5% missing data or less. See, FIG. 1

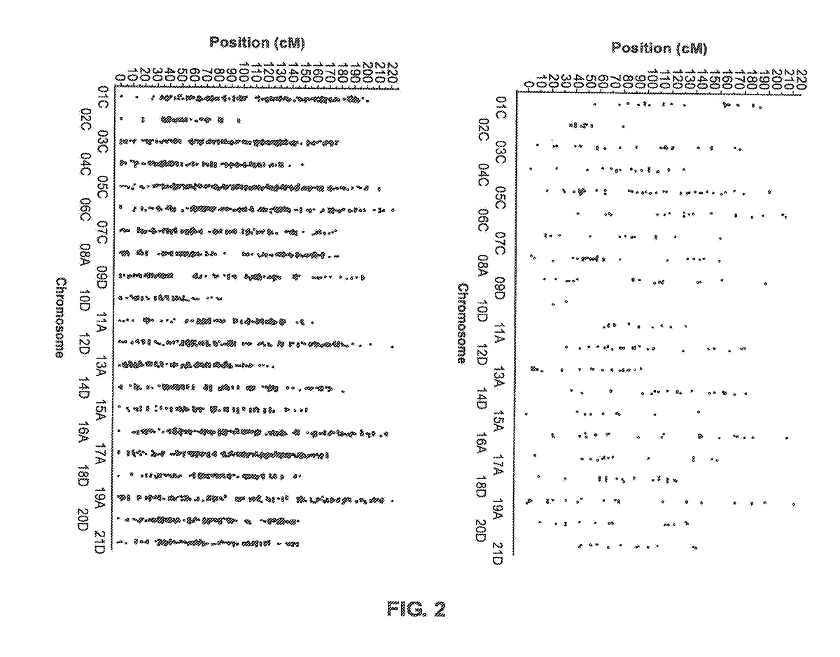

Of the 136 calculated linkage blocks, 115 blocks contained at least 1 locus in which the BAD assay was able to successfully determine a polymorphic nucleotide. Seven of the 21 chromosomes were completely saturated, while all but two of the remaining chromosomes were at least 90% saturated. See, FIG. 2.

Cluster analysis of the highly related breeding lines tested revealed variations based on the marker datasets used. The genetic relationships between the 15 tested lines were significantly narrower when using the BAD assay than GBS. In addition, only two lines did not cluster with other lines based on the data obtained using the BAD assay, as compared to four when using GBS. Thus, cluster analysis results from data using the BAD assay appeared to fit the known structure of the breeding lines better than the cluster analysis results from data obtained using GBS.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

“Include,” “including,” or like terms means encompassing but not limited to, that is, including and not exclusive.

The implementations described above and other implementations are within the scope of the following claims. One skilled in the art will appreciate that the present disclosure can be practiced with embodiments other than those disclosed. The disclosed embodiments are presented for purposes of illustration and not limitation.

Lengthy table referenced here
US20170204474A1-20170720-T00001
Please refer to the end of the specification for access instructions.

Lengthy table referenced here
US20170204474A1-20170720-T00002
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20170204474A1). An electronic copy of the table will also be available from the USPTO upon request and payment of the fee set forth in 37 CFR 1.19(b)(3).

What is claimed is:

1. A method comprising:
a) providing a plurality of primer pairs, each primer pair of the plurality of primer pairs being configured to amplify a unique locus in a pseudo genome, the pseudo genome comprising at least a portion of a full genome and including a plurality of identified polymorphisms;
b) for each of a plurality of individuals, subjecting a sample comprising genomic DNA (gDNA) to a polymerase chain reaction using at least a portion of the plurality of primer pairs to obtain a pool of amplified products for each of the plurality of individuals;
c) determining sequences for each of the amplified products; and
d) determining an allele composition of each individual of the plurality of individuals based on the determined sequences, wherein each unique locus has at least two possible alleles.

2. The method of claim 1, wherein the at least a portion of the plurality of primer pairs comprises at least 100 primer pairs.

3. The method of claim 1, further comprising attaching a tag to at least one end of the amplified products in each pool to produce a plurality of coded pools, the tag being different for each pool of amplified products, and combining the coded pools prior to determining the sequences of the amplified products.

4. The method of claim 1, wherein the identified polymorphisms are single nucleotide polymorphisms (SNPs).

5. The method of claim 1, wherein the determined sequences comprise 1 to 12 polymorphic nucleotides per unique locus.

6. The method of claim 5, wherein the average number of polymorphic nucleotides per unique locus of the determined sequences is greater than the average number of identified polymorphisms per unique locus.

7. The method of claim 1, further comprising determining the zygosity of each individual of the plurality of individuals at each unique locus.

8. The method of claim 1, further comprising:
e) determining linkage disequilibrium of the identified polymorphisms using the at least a portion of the plurality of primer pairs;
f) selecting a subset of the primer pairs based on the linkage disequilibrium; and
g) using the subset of primer pairs to obtain the pool of amplified products.

9. The method of claim 1, further comprising providing a plurality of additional primer pairs, the plurality of additional primer pairs configured to amplify a plurality of additional loci on one or more selected genes in the full genome and outside of the pseudo genome.

10. The method of claim 9, wherein the plurality of additional loci are on two or more genes associated with a biosynthetic pathway.

11. The method of claim 10, wherein the biosynthetic pathway is a beta-glucan synthesis pathway.

12. The method of claim 9, wherein the plurality of additional loci are on an avenanthramide gene.

13. The method of claim 1, wherein the full genome is representative of a species.

14. The method of claim 1, wherein the full genome is representative of a selected population within a species.

15. The method of claim 14, wherein the selected population is a breed or strain within a species.

16. The method of claim 1, wherein the full genome is an oat, a broccoli, or a maize genome.

17. The method of claim 1, wherein the pseudo genome contains at least a portion of each chromosome in the full genome.

18. The method of claim 1, wherein the pseudo genome is a transcriptome.

19. A method for producing a library of primers for genotyping a plurality of individuals, comprising:
a) providing a pseudo genome sequence, the pseudo genome sequence being derived from a full genome sequence and including a plurality of identified polymorphisms;
b) designing primer pairs configured to amplify a locus containing each identified polymorphism, each primer pair configured to amplify a unique locus in the pseudo genome sequence;
c) synthesizing at least a portion of the primer pairs;
d) determining linkage disequilibrium of the identified polymorphisms using the primer pairs;
e) selecting a subset of the primer pairs based on the linkage disequilibrium; and
f) using the subset of primer pairs to produce the library.

20. The method of claim 19, wherein the library comprises at least 100 primer pairs.

21. The method of claim 19, wherein the identified polymorphisms are SNPs.

22. The method of claim 19, further comprising providing a plurality of additional primer pairs, the plurality of additional primer pairs configured to amplify a plurality of additional loci on one or more selected genes in the full genome and outside of the pseudo genome.

23. The method of claim 22, wherein the plurality of additional loci are on two or more genes associated with a biosynthetic pathway.

24. The method of claim 23, wherein the biosynthetic pathway is a beta-glucan synthesis pathway.

25. The method of claim 24, wherein the plurality of additional loci are on an avenanthramide gene.

26. The method of claim 19, wherein the full genome is representative of a species.

27. The method of claim 19, wherein the full genome is representative of a selected population within a species.

28. The method of claim 27, wherein the selected population is a breed or strain within a species.

29. The method of claim 19, wherein the pseudo genome sequence contains at least a portion of each chromosome in the full genome sequence.

30. The method of claim 19, wherein the pseudo genome sequence is a transcriptome sequence.

31. The method of claim 19, wherein the full genome sequence is an oat, a broccoli, or a maize genome sequence.
